(19) United States
(12) Patent Application Publication
(10) Pub. No.: US 2022/0323325 A1
(43) Pub. Date: Oct. 13, 2022

(54) COMPOSITIONS COMPRISING DISSUBSTITUTE TEREPHTHALATES AND ALSO LIGHT PROECTION FILTER SUBSTANCES, DEODORANT AND/OR ANTIPERSPIRANT ACTIVE INGREDIENTS

(71) Applicant: Evonik Operations GmbH, Essen (DE)

(72) Inventors: Matthias Mentel, Dortmund (DE); Juergen Meyer, Essen (DE); Achim Friedrich, Hattingen (DE)

(73) Assignee: Evonik Operations GmbH, Essen (DE)

(21) Appl. No.: 17/753,446

(22) PCT Filed: Aug. 27, 2020
(86) PCT No.: PCT/EP2020/073921

§ 371 Date: Mar. 3, 2022

(30) Foreign Application Priority Data
Sep. 5, 2019 (EP) 19195485.8

(51) Int. Cl. A61K 8/37; A61Q 15/00; A61Q 17/04

(52) U.S. Cl. CPC A61K 8/37; A61Q 15/00; A61Q 17/04

(57) ABSTRACT

Compositions containing disubstituted terephthalates are useful. The compositions may also contain light protection filter substances, deodorant and/or antiperspirant active ingredients.

### FIELD OF THE INVENTION

The invention relates to compositions comprising disubstituted terephthalates and also light protection filter substances, deodorant and/or antiperspirant active ingredients.

### PRIOR ART

In the case of frequent or large area applications of sunscreen preparations, it cannot be excluded that high dose UV light protection filter substances are applied to the skin in gram amounts. These amounts of UV light protection filter substances have to be incorporated, dissolved homogeneously and stably, into the formulation.

To dissolve these UV light protection filter substances, use is often made of oily components which have a good dissolution capacity for UV light protection filter substances. One compound class used with good dissolution capacity are aliphatic benzoic esters. A typical representative of this compound class is C12-C15 alkyl benzoate, which has been established worldwide as an ingredient for sunscreen formulations.

EP2165696 discloses the use of phenoxyalkyl esters of C6-C12 alkyl- and cycloalkylcarboxylic acids for preparing a cosmetic, dermatological or pharmaceutical formulation and use as solubilizer or solvent for at least one UV light protection filter substance.

EP2062566 discloses the use of isononyl benzoate for preparing a cosmetic, dermatological or pharmaceutical formulation and use as solubilizer or solvent for at least one UV light protection filter substance.

The need for highly concentrated sunscreen formulations with respect to the UV light protection filter substances is due to increased health awareness and still growing recreational behaviour of consumers.

The object of the invention was to provide substances having good dissolution capacity for UV light protection filter substances but also for deodorant and antiperspirant active ingredients.

### DESCRIPTION OF THE INVENTION

Surprisingly, it has been found that disubstituted terephthalates have excellent dissolution properties for UV light protection filter substances but also for deodorant and antiperspirant active ingredients.

The present invention therefore relates to compositions comprising disubstituted terephthalates and also light protection filter substances, deodorant and/or antiperspirant active ingredients as described in claim 1.

The invention further relates to the use of disubstituted terephthalates as solubilizers or solvents in a cosmetic or pharmaceutical composition.

One advantage of the present invention is that the disubstituted terephthalates readily dissolve light protection filters for different UV ranges over a broad temperature range.

A further advantage of the present invention is that sunscreen formulations comprising disubstituted terephthalates, when applied to the skin, are characterized by a relatively low oily and well-absorbed skin feel compared to sunscreen formulations comprising C12-15 alkyl benzoates.

A further advantage of the present invention is that sunscreen formulations comprising disubstituted terephthalates have higher storage stabilities, and thereby have a longer shelf life, compared to sunscreen formulations comprising C12-15 alkyl benzoates.

Another advantage of the present invention is that the disubstituted terephthalates stably disperse inorganic pigments and are therefore also suitable for preparing compositions comprising deodorant and/or antiperspirant active ingredients.

A further advantage of the present invention is that the disubstituted terephthalates have good spreadability and thus achieve homogeneous distribution of the light protection factors on the skin.

Unless stated otherwise, all percentages (%) given are percentages by mass.

The present invention therefore relates to a composition comprising

A) at least one substance of the general formula (I)

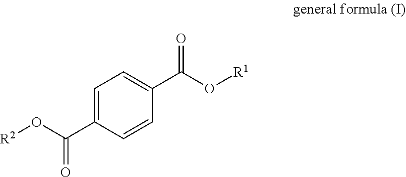

where R¹ and R² are each independently, the same or different, selected from the group of organic radicals comprising 2 to 18 carbon atoms, preferably aliphatic, especially alkyl radicals, particularly preferably alkyl radicals comprising 4 to 8 carbon atoms; and

B) at least one substance selected from the group of UV light protection filter substances, deodorant and antiperspirant active ingredients, especially UV light protection filter substances.

Substances of the general formula (I) are synthetically accessible, as described, for example, in EP3351526.

In accordance with the invention, R¹ and R² are each independently, the same or different, selected from the group of branched alkyl radicals, particularly preferably comprising 4 to 8 carbon atoms, especially isopentyl.

The UV light protection filter substances present can be, for example, organic substances which are capable of absorbing ultraviolet radiation and emitting again the absorbed energy in the form of longer-wave radiation, for example heat.

UVB filters can be oil-soluble or water-soluble. Oil-soluble UVB light protection filters include, for example:

3-benzylidenecamphor derivatives, e.g. 3-(4-methylbenzylidene)camphor (INCI: 4-Methylbenzylidene Camphor), 4-aminobenzoic acid derivatives such as e.g. 2-ethylhexyl 4-(dimethylamino)benzoate (INCI: Ethylhexyl Dimethyl PABA), 2-hydroxyethyl 4-{bis[2-(2-hydroxyethoxy)ethyl]amino}benzoate (INCI: PEG-25 PABA), esters of cinnamic acid such as e.g. 2-ethylhexyl 4-methoxycinnamate (INCI; Ethylhexyl Methoxycinnamate), isopentyl 4-methoxycinnamate (INCI: Isoamyl p-Methoxycinnamate), 2-ethylhexyl 2-cyano-3,3-diphenylacrylate (INCI: Octocrylene), esters of salicylic acid such as e.g. 2-ethylhexyl salicylate (INCI: Ethylhexyl Salicylate), homomenthyl salicylate (INCI: Menthyl Salicylate), 3,3,5-trimethylcyclohexyl salicylate (INCI: Homosalate), 2-hydroxybenzoic acid 2,2′,2″-nitrilotriethanol (1:1) (INCI: TEA-Salicylate), derivatives of benzophenone such as e.g. 2-hydroxy-4-methoxybenzophenone (INCI: Benzophenone-3), 2,2′-dihydroxy-4-methoxybenzophenone (INCI: Benzophenone-8), 2-hydroxy-4-methoxy-4′-methylbenzophenone (INCI: Benzophenone-10), triazine derivatives such as e.g. tris(2-ethylhexyl) 4,4′,4″-(1,3,5-triazine-2,4,6-triyltriimino)tribenzoate (INCI: Ethylhexyl Triazone, obtainable for example under the trade name Uvinul T 150 from BASF), iscotrizinole (INCI: Diethylhexyl Butamido Triazone) and 2,4,6-tri(4-biphenylyl)-1,3,5-triazine (INCI: Tris-Biphenyl Triazine). Another UVB filter is 3-(4-(2,2-bisethoxycarbonylvinyl)phenoxy)propenyl)methoxysiloxane/dimethylsiloxane copolymer (INCI: Polysilicone-15), which can be obtained for example under the trade name Parsol SLX. Possible water-soluble UVB light protection filters are, for example: salts of 2-phenylbenzimidazole-5-sulfonic acid such as the alkali metal, alkaline earth metal, ammonium, alkylammonium, alkanolammonium and glucammonium salts thereof and also the sulfonic acid itself with the INCI name Phenylbenzimidazole Sulfonic Acid, sulfonic acid derivatives of benzophenone such as e.g. 5-benzoyl-4-hydroxy-2-methoxybenzenesulfonic acid (NCI: Benzophenone-4) and salts thereof, benzensulfonic acid derivatives of 3-benzylidenecamphor such as e.g. 4-[(E)-(4,7,7-trimethyl-3-oxobicyclo[2.2.1]hept-2-ylidene)methyl]benzenesulfonic acid (INCI: Benzylidene Camphor Sulfonic Acid) and salts thereof.

Typical suitable oil-soluble broadband UVA light protection filters are, for example, derivatives of benzoylmethane such as for example 1-(4-methoxyphenyl)-3-[4-(2-methyl-2-propanyl)phenyl]-1,3-propanedione (INCI: Butyl Methoxydibenzoylmethane), 1-(4-isopropylphenyl)-3-phenyl-1,3-propanedione (INCI: Isopropyl Dibenzoylmethane), triazine derivatives such as e.g. 2,2′-[6-(4-methoxyphenyl)-1,3,5-triazine-2,4-diyl]bis{5-[(2-ethylhexyl)oxy]phenol} (INCI: Bis-Ethylhexyloxyphenol Methoxyphenyl Triazine, obtainable for example under the trade name Tinosorb S from BASF), N,N′-bis[4-[5-(1,1-dimethylpropyl)-2-benzoxazolyl]phenyl]-N″-(2-ethylhexyl)-1,3,5-triazine-2,4,6-triamine (INCI: Ethylhexyl Bis-Isopentylbenzoxazolylphenyl Melamine, obtainable under the trade name Uvasorb K2A from 3V Sigma) and derivatives of benzophenone such as e.g. hexyl 2-(4-diethylamino-2-hydroxybenzoyl)benzoate (INCI: Diethylamino Hydroxybenzoyl Hexyl Benzoate).

Possible water-soluble broadband UVA light protection filters are, for example: 3,3′-(1,4-phenylenedimethylene)bis(7,7-dimethyl-2-oxo-bicyclo[2.2.1]hept-1-ylmethanesulfonic acid) and salts thereof, especially the corresponding sodium, potassium or triethanolammonium salt, which is also referred to as benzene-1,4-di(2-oxo-3-bornylidenemethyl-10-sulfonic acid) and has the INCI name Terephthalylidene Dicamphor Sulfonic Acid (obtainable under the trade name Mexoryl SX), 2,2′-(1,4-phenylene)bis(6-sulfa-1H-benzimidazole-4-sulfonic acid) and salts thereof, the corresponding sodium, potassium or triethanolammonium salts, e.g. disodium 2,2′-(1,4-phenylene)bis(6-sulfo-1H-benzimidazol-4-sulfonate) with the INCI name Disodium Phenyl Dibenzimidazole Tetrasulfonate, trade name e.g. Neo Heliopan AP.

Further broadband UVA filters are, e.g. 2,2′-methylenebis[6-(2H-benzotriazol-2-yl)-4-(2,4,4-trimethyl-2-pentanyl)phenol] (INCI: Methylene Bis-Benzotriazolyl Tetramethylbutylphenol, obtainable for example under the trade name Tinosorb M from BASF), 2-(2H-benzotriazol-2-yl)-4-methyl-6-[2-methyl-3-[1,3,3,3-tetramethyl-1-[(trimethylsilyl)oxy]disiloxanyl]propyl]phenol (INCI: Drometrizole Trisiloxane, trade name: Mexoryl XL), (1R,2S,5R)-2-isopropyl-5-methylcyclohexyl 2-aminobenzoate (INCI: Menthyl Anthranilate) and 2-ethoxyethyl (2E)-3-(4-methoxyphenyl)acrylate (INCI: Cinoxate).

The UV filters can also obviously be present in mixtures in the compositions according to the invention.

Besides the soluble substances specified, insoluble pigments are also suitable for this purpose, namely finely dispersed metal oxides or salts, such as for example titanium dioxide, zinc oxide, iron oxide, aluminium oxide, cerium oxide, zirconium oxide, silicates (talc), barium sulfate and zinc stearate. The particles here should have an average diameter of less than 100 nm, e.g. between 5 and 50 nm and in particular between 15 and 30 nm. They can have a spherical shape, although it is also possible to use those particles which have an ellipsoidal shape or a shape which deviates in some other way from the spherical form. Also possible, however, are micronized organic pigments such as for example 2,2′-methylenebis[6-(2H-benzotriazol-2-yl)-4-(2,4,4-trimethyl-2-pentanyl)phenol] (INCI: Methylene Bis-Benzotriazolyl Tetramethylbutyiphenol, obtainable for example under the trade name Tinosorb M from BASF) having a particle size of <200 nm, which can be obtained, for example, as a 50% aqueous dispersion.

In addition, further suitable UV light protection filters can be found in the overview by P. Finkel in SÖFW-Journal 122, 543 (1996) or the chapter The Chemistry of Ultraviolet Filters by N. A. Shaath in Principles and Practice of Photoprotection, S. Q. Wang, H. W. Lim (eds.), Springer International Publishing Switzerland 2016.

It is preferred in accordance with the invention that component B) of the composition according to the invention is selected from at least two, preferably at least three, particularly preferably at least four, selected from the group of UV light protection filter substances.

It is preferred in accordance with the invention that component B) of the composition according to the invention is selected from the group of organic UV light protection filter substances, especially from the group of triazine derivatives.

It is particularly preferred in accordance with the invention that component B) of the composition according to the invention is selected from the group of UV light protection filter substances comprising, preferably consisting of,

butyl methoxydibenzoylmethane,

ethylhexyl triazone,

bis-ethylhexyloxyphenol methoxyphenyl triazine,

diethylhexyl butamido triazone,

diethylamino hydroxybenzoyl hexyl benzoate,

benzophenone-3,

4-methylbenzylidene camphor.

It is furthermore preferred in accordance with the invention that component B) of the composition according to the invention is selected from the group of deodorant and antiperspirant active ingredients comprising, preferably consisting of, astringents, preferably aluminium salts, zinc salts and aluminium-zirconium complexes. From this group, particular preference is given to the presence of basic aluminium chlorides such as aluminium chlorohydrate (“ACH”) or aluminium-zirconium-glycine salts (“ZAG”),

A preferred composition according to the invention is characterized in that component A) is present in an amount of 0.1% by weight to 60% by weight, preferably 0.5% by weight to 30% by weight, particularly preferably 1% by weight to 20% by weight, and component B) is present in an amount of 0.1% by weight to 50% by weight, preferably 1% by weight to 40% by weight, particularly preferably 3% by weight to 30% by weight.

The compositions according to the invention can comprise e.g. at least one further, additional component selected from the group of
emollients,
co-emulsifiers,
thickeners/viscosity regulators/stabilizers,
antioxidants,
hydrotropes (or polyols),
solids and fillers,
pearlescence additives,
insect repellents,
self-tanning agents,
preservatives,
conditioning agents,
perfumes,
dyes,
cosmetic active ingredients,
care additives,
superfatting agents,
solvents.

Substances which can be used as exemplary representatives of the individual groups are known to those skilled in the art and can be found for example in German application DE 102008001788.4. This patent application is hereby incorporated as reference and thus forms part of the disclosure. As regards further optional components and the amounts used of these components, reference is made expressly to the relevant handbooks known to the person skilled in the art, e.g. K. Schrader, “Grundlagen and Rezepturen der Kosmetika [Fundamentals and formulations of cosmetics]”, 2nd edition, pages 329 to 341, Hüthig Buch Verlag Heidelberg.

The amounts of the particular additives are determined by the intended use.

Typical boundary formulations for the respective applications are known prior art and are contained for example in the brochures of the manufacturers of the particular base and active ingredients. These existing formulations can generally be adopted unchanged. However, if required, for adjustment and optimization, the desired modifications can be undertaken by simple tests without complication.

Compositions according to the invention can be used, for example, as skin care, facial care, head care, body care, intimate care, foot care, hair care, nail care, dental care or oral care products.

Compositions according to the invention can be used, for example, in the form of an emulsion, a suspension, a solution, a cream, a salve, a paste, a gel, an oil, a powder, an aerosol, a stick, a spray, a cleansing product, a make-up or sunscreen preparation or a facial toner.

The present invention further relates to the use of at least one substance of the general formula (I)

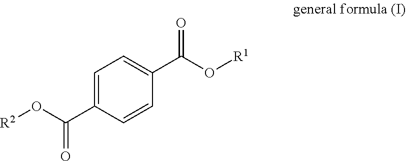

where R¹ and R² are each independently, the same or different, selected from the group of organic radicals comprising 2 to 18 carbon atoms, preferably aliphatic, especially alkyl radicals, particularly preferably alkyl radicals comprising 4 to 8 carbon atoms;

as solubilizer or solvent in a cosmetic or pharmaceutical composition, particularly as solubilizer or solvent for at least one substance selected from the group of UV light protection filter substances, deodorant and antiperspirant active ingredients, especially UV light protection filter substances.

In the use according to the invention, preference is given to using the components specified above as components preferably present in the context of the compositions according to the invention.

The examples which follow describe the present invention by way of example, without any intention of restricting the invention, the scope of which is apparent from the entirety of the description and the claims, to the embodiments cited in the examples.

The following figures are part of the examples:

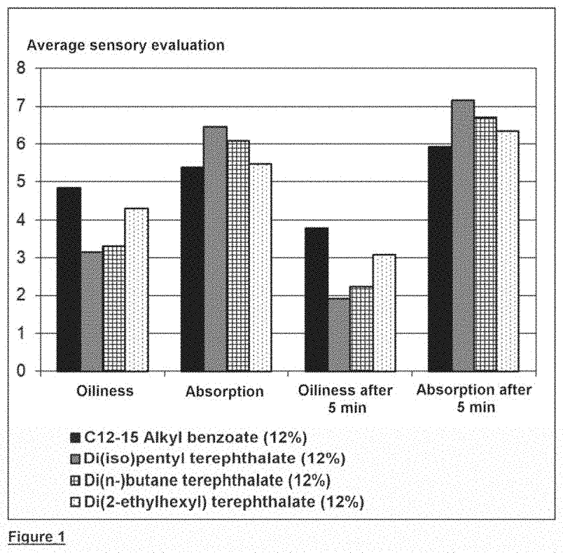

FIG. 1: Sensory evaluation of emulsions

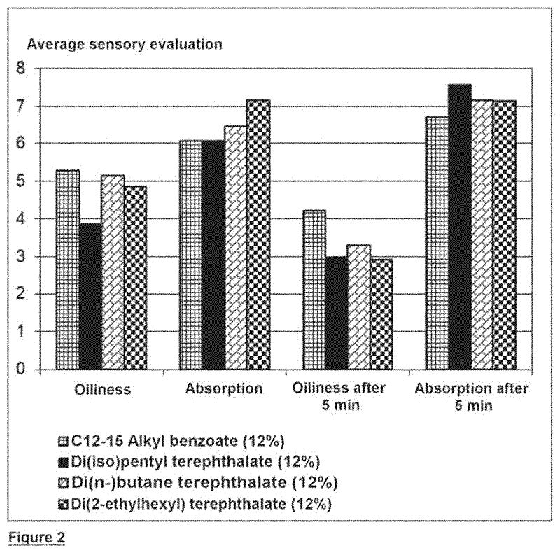

FIG. 2: Sensory evaluation of emulsions

### EXAMPLES

### Example 1: Dissolution Capacity of UV Light Protection Filters

To test the dissolution capacity of UV light protection filters, three representative UVA or UVB light protection filters were selected representatively. These are:
Butyl Methoxydibenzoylmethane (BMDM)
Ethylhexyl Triazone (EHT)
Bis-Ethylhexyloxyphenol Methoxyphenyl Triazine (BEMT).

To determine the dissolution capacity for these three UV light protection filters, a certain amount (50 g) of one of the compounds according to the invention was initially charged in each case and temperature-controlled at 22° C. A percentage by weight of a UV light protection filter was added and stirred until this amount had fully and homogeneously dissolved. This procedure was repeated until the maximal soluble amount of the UV light protection filter had been exceeded. For complete dissolution at higher concentrations, a longer stirring time of several hours is often required. If the maximum concentration had been roughly determined in this manner, a fine determination of the concentration range was repeated around this maximum concentration using smaller weighed amounts of the UV light protection filter.

### C12-15 Alkyl Benzoate and 2-Phenoxyethyl Octanoate were Used as Reference

| | BMDM | EHT | BEMT |
|---|---|---|---|
| C12-15 Alkyl | 13% | 4% | 9% |
| Benzoate | | | |
| 2-Phenoxyethyl | 18% | 6% | 11% |
| octanoate | | | |
| Di(iso)pentyl | 22% | 14% | 13% |
| terephthalate | | | |
| Di(2-ethylhexyl) | 20% | 7% | 12% |
| terephthalate | | | |
| Di(n-)butane | 23% | 18% | 23% |
| terephthalate | | | |

The dialkyl terephthalates used dissolve the UV light protection filters investigated better than C12-15 alkyl benzoate and 2-phenoxyethyl octanoate.

In particular, the dialkyl terephthalates used are characterized by excellent properties in the dissolution capacity for EHT.

### Example 2: Sensory Evaluation of a Sunscreen Formulation SPF 50, PA++++ Comprising Dialkyl Terephthalates

The formulations shown in Table 1 were prepared. The influence on skin feel of the formulations was investigated by a test panel. Thirteen people each applied a defined amount of approx. 25 μl of both formulations to a defined test field on the inside of the forearm without knowing the composition of the formulations. The formulations were spread with a finger by circular movements in the test field. The parameters oiliness and absorption were assessed during spreading of the sample. After absorption was complete, there was a pause and, after 5 minutes, the skin feel on the test field was re-assessed.

TABLE 1
Oil-in-water emulsions. Figures in percentage by weight Classical lotion preparation.
Phases A and B are heated to 85° C., phase B is added to A and homogenized for 90 s with an
UltraTurrax (20 500 rpm). At 50° C., phase C is added and homogenized for 30 s with the
UltraTurrax (20 500 rpm). At 35° C., phase D and E is added and homogenized for 30 s with the
UltraTurrax (13 500 rpm). The pH is adjusted to 6.5-7.0 with tromethamine
(tris(hydroxymethyl)aminomethane, 30% in water).

| | ST 18/18 | 22 | 23 | 32 | 33 |
|---|---|---|---|---|---|
| A | TEGO ® Care PBS 6 (Polyglyceryl-6 Stearate; | 4.00 | 4.00 | 4.00 | 4.00 |
| | Polyglyceryl-6 Behenate) | | | | |
| | TEGIN ® M Pellets (Glyceryl Stearate) | 0.25 | 0.25 | 0.25 | 0.25 |
| | TEGO ® Alkanol 1618 (Cetearyl Alcohol) | 0.25 | 0.25 | 0.25 | 0.25 |
| | C12-15 Alkyl Benzoate | 12.00 | | | |
| | Di(iso)pentyl terephthalate | | 12.00 | | |
| | Di(n-)butane terephthalate | | | 12.00 | |
| | Di(2-ethylhexy) terephthalate | | | | 12.00 |
| | Bis-Ethylhexyloxyphenol Methoxyphenyl Triazine | 6.70 | 6.70 | 6.70 | 6.70 |
| | (Tinosorb S, BASF) | | | | |
| | Butyl Methoxydibenzoylmethane | 2.50 | 2.50 | 2.50 | 2.50 |
| | Diethylamino Hydroxybenzoyl Hexyl Benzoate | 2.50 | 2.50 | 2.50 | 2.50 |
| | Diethylhexyl Butamido Triazone | 2.00 | 2.00 | 2.00 | 2.00 |
| | Ethylhexyl Triazone (Uvinul T 150, BASF) | 2.00 | 2.00 | 2.00 | 2.00 |
| | dermofeel ® Toco 70 non GMO (Tocopherol; Helianthus | 0.20 | 0.20 | 0.20 | 0.20 |
| | Annuus (Sunflower) Seed Oil) | | | | |
| B | Water | 52.75 | 52.75 | 52.75 | 52.75 |
| | Glycerol | 3.00 | 3.00 | 3.00 | 3.00 |
| | EDTA | 0.05 | 0.05 | 0.05 | 0.05 |
| C | Xanthan Gum (Keltrol CG-SFT, CP Kelco) | 0.50 | 0.50 | 0.50 | 0.50 |
| D | Phenylbenzimidazole Sulfonic Acid | 2.00 | 2.00 | 2.00 | 2.00 |
| | Tromethamine | 0.88 | 0.88 | 0.88 | 0.88 |
| | Water | 7.12 | 7.12 | 7.12 | 7.12 |
| E | Verstatil ® BOB (Benzyl Alcohol; Caprylyl Glycol; Benzoic | 1.20 | 1.20 | 1.20 | 1.20 |
| | Acid) | | | | |
| Z | Perfume | q.s. | q.s. | q.s. | q.s. |

FIG. 1 shows that the formulation with dialkyl terephthalates is less oily and shows better absorption after 5 min compared to C12-15 alkyl benzoate.

### Example 3: Sensory Evaluation of a Sunscreen Formulation SPF 50, PA++++ Comprising Dialkyl Terephthalates

The formulations shown in Table 2 were prepared. The influence on skin feel of the formulations was investigated by a test panel. Fourteen people each applied a defined amount of approx. 25 μl of both formulations to a defined test field on the inside of the forearm without knowing the composition of the formulations. The formulations were spread with a finger by circular movements in the test field. The parameters oiliness and absorption were assessed during spreading of the sample. After absorption was complete, there was a pause and, after 5 minutes, the skin feel on the test field was re-assessed.

TABLE 2
Oil-in-water emulsions. Figures in percentage by weight. Classical
lotion preparation. Phases A and B are heated to 85° C.,
phase B is added to A and homogenized for 90 s with an UltraTurrax
(20 500 rpm). At 50° C., phase C is added and homogenized
for 30 s with the UltraTurrax (20 500 rpm). At 35° C.,
phase D and E is added and homogenized for 30 s with the UltraTurrax
(13 500 rpm). The pH is adjusted to 6.5-7.0 with tromethamine
(tris(hydroxymethyl)aminomethane, 30% in water).

| | ST 18/18 | 25 | 26 | 34 | 35 |
|---|---|---|---|---|---|
| A | TEGO ® Care PBS 6 (Polyglyceryl-6 | 4.00 | 4.00 | 4.00 | 4.00 |
| | Stearate; Polyglyceryl-6 Behenate) | | | | |
| | TEGIN ® M Pellets (Glyceryl Stearate) | 0.25 | 0.25 | 0.25 | 0.25 |
| | TEGO ® Alkanol 1618 (Cetearyl | 0.25 | 0.25 | 0.25 | 0.25 |
| | Alcohol) | | | | |
| | C12-15 Alkyl Benzoate | 9.00 | | | |
| | Di(iso)pentyl terephthalate | | 9.00 | | |
| | Di(n-)butane terephthalate | | | 9.00 | |
| | Di(2-ethylhexy) terephthalate | | | | 9.00 |
| | Bis-Ethylhexyloxyphenol | 7.00 | 7.00 | 7.00 | 7.00 |
| | Methoxyphenyl Triazine | | | | |
| | (Tinosorb S, BASF) | | | | |
| | Butyl Methoxydibenzoylmethane | 2.00 | 2.00 | 2.00 | 2.00 |
| | Diethylamino Hydroxybenzoyl Hexyl | 2.00 | 2.00 | 2.00 | 2.00 |
| | Benzoate | | | | |
| | Diethylhexyl Butamido Triazone | 1.00 | 2.00 | 1.00 | 2.00 |
| | Ethylhexyl Triazone (Uvinul T 150, | 2.00 | 2.00 | 2.00 | 2.00 |
| | BASF) | | | | |
| | Ethylhexyl Salicylate | 4.50 | 4.50 | 4.50 | 4.50 |
| | dermofeel ® Toco 70 non GMO | 0.20 | 0.20 | 0.20 | 0.20 |
| | (Tocopherol; Helianthus Annuus | | | | |
| | (Sunflower) Seed Oil) | | | | |
| B | Water | 52.95 | 52.95 | 52.95 | 52.95 |
| | Glycerol | 3.00 | 3.00 | 3.00 | 3.00 |
| | EDTA | 0.05 | 0.05 | 0.05 | 0.05 |
| C | Xanthan Gum (Keltrol CG-SFT, | 0.50 | 0.50 | 0.50 | 0.50 |
| | CP Kelco) | | | | |
| D | Phenylbenzimidazole Sulfonic Acid | 2.00 | 2.00 | 2.00 | 2.00 |
| | Tromethamine | 0.88 | 0.88 | 0.88 | 0.88 |
| | Water | 7.12 | 7.12 | 7.12 | 7.12 |
| E | Verstatil ® BOB (Benzyl Alcohol; | 1.20 | 1.20 | 1.20 | 1.20 |
| | Caprylyl Glycol; Benzoic Acid) | | | | |
| Z | Perfume | q.s. | q.s. | q.s. | q.s. |

FIG. 2 shows that the formulation with dialkyl terephthalates is less oily and shows better absorption after 5 min compared to C12-15 alkyl benzoate.

### Example 4: Storage Stability of a Sunscreen Formulation SPF 50, PA Comprising Dialkyl Terephthalates

The oil-in-water sunscreen emulsions shown in Table 1 were prepared. The viscosities of the formulations are comparable (7-8, or 9 Pa·s; Brookfield viscosity, measured with spindle 4, 5 rpm).

All the formulations are low-viscosity lotions. The formulations were stored at 40° C. and 45° C. for three months. After three months, both samples stored at 40° C. and at 45° C. showed significant oil separation of the formulation comprising C12-15 alkyl benzoate, but not in the formulations according to the invention comprising dialkyl terephthalates.

### Example 5: Formulation Examples

The formulation examples listed below are listed using di(iso)pentyl terephthalate.

All the following listed formulation examples are in each case additionally formulated, instead of di(iso)pentyl terephthalate, with di(neo)pentyl terephthalate, di(n-)pentyl terephthalate, (iso/neo)pentyl terephthalate, (iso/n-)pentyl terephthalate, (neo/n-)pentyl terephthalate, di(iso)hexyl terephthalate, di(2-ethylhexyl) terephthalate, di(n-)butane terephthalate, di(2-methylpropane) terephthalate and n-butane/2-methylpropane terephthalate.

### Sun Care Spray SPF 30

| Phase | Raw material | w/w % |
|---|---|---|
| A | TEGO ® Care PBS 6 (Polyglyceryl-6 Stearate, | 3.00 |
| | Polyglyceryl-6 Behenate) | |
| | Di(iso)pentyl terephthalate | 3.00 |
| | Acrylates/Octylacrylamide Copolymer | 0.50 |
| | Bis-ethylhexyloxyphenol Methoxyphenyl Triazine | 3.00 |
| | (Tinosorb S, BASF) | |
| | Butyl Methoxydibenzoylmethane | 2.00 |
| | Homosalate | 4.00 |
| | Ethylhexyl Salicylate | 4.00 |
| | Octocrylene | 4.00 |
| | Diethylhexyl 2,6-Naphthalate | 2.00 |
| B | Water | to 100 |
| | Gellan Gum (Kelcogel CG-HA, CP Kelco) | 0.03 |
| | EDTA | 0.05 |
| | Glycerol | 3.00 |
| C | Phenylbenzimidazole Sulfonic Acid | 2.00 |
| | Tromethamine | 0.88 |
| | Water | 7.12 |
| D | Acrylates/C10-30 Alkyl Acrylate Crosspolymer | 0.20 |
| | Water | 9.80 |
| E | Tromethamine (Trisaminomethane, 30% in water) | q.s. |
| F | Phenoxyethanol, Ethylhexylglycerin (Euxyl PE 9010, | 0.70 |
| | Schülke & Mayr GmbH) | |

### Oil Release Sun Care Lotion SPF 50

| Phase | Raw material | w/w % |
|---|---|---|
| A | TEGO ® Care PBS 6 (Polyglyceryl-6 Stearate, | 3.00 |
| | Polyglyceryl-6 Behenate) | |
| | Di(iso)pentyl terephthalate | 15.00 |
| | TEGOSOFT ® DEC (Diethylhexyl Carbonate) | 10.00 |
| | TEGOSOFT ® XC (Phenoxyethyl Caprylate) | 10.00 |
| | Styrene/Acrylates Copolymer | 1.00 |
| | Diethylamino Hydroxybenzoyl Hexyl Benzoate | 10.00 |
| | (Uvinul A plus, BASF) | |
| | Ethylhexyl Methoxycinnamate | 10.00 |
| | Bis-Ethylhexyloxyphenol Methoxyphenyl Triazine | 5.00 |
| | (Tinosorb S, BASF) | |
| | Ethylhexyl Salicylate | 5.00 |
| | Homosalate | 5.00 |
| | TEGO ® Feel C 10 (Cellulose) | 1.00 |
| B | Water | to 100 |
| | Glycerol | 3.00 |
| C | Phenoxyethanol, Ethylhexylglycerin (Euxyl PE 9010, | 0.70 |
| | Schülke & Mayr GmbH) | |
| D | Citric Acid (10% in water) | q.s. |

### Sun Care Spray SPF 30

| Phase | Raw material | w/w % |
|---|---|---|
| A | Di(iso)pentyl terephthalate | 20.00 |
| | C12-15 Alkyl Benzoate | 8.00 |
| | TEGOSOFT ® APM (PPG-3 Myristyl Ether) | 8.00 |
| | VA/Butyl Maleate/lsobornyl Acrylate Copolymer (and) | 1.00 |
| | Alcohol | |
| | TEGO ® Xymenynic (Caprylic/Capric Triglyceride; | 2.00 |
| | Xymenynic Acid) | |
| | Octocrylene | 6.80 |
| | Ethylhexyl Salicylate | 2.50 |
| | Butyl Methoxydibenzoylmethane | 5.00 |
| | Bis-Ethylhexyloxyphenol Methoxyphenyl Triazine | 3.00 |
| | (Tinosorb S, BASF SE) | |
| | Diethylhexyl Butamido Triazone | 1.20 |
| B | Alcohol | to 100 |

### Transparent Sun Care Spray SPF 25

| Phase | Raw material | w/w % |
|---|---|---|
| A | Di(iso)pentyl terephthalate | 12.00 |
| | TEGOSOFT ® APM (PPG-3 Myristyl Ether) | 3.00 |
| | TEGOSOFT ® GC (PEG-7 Glyceryl Cocoate) | 1.50 |
| | TEGO ® Turmerone (Curcuma Longa (Turmeric) | 0.50 |
| | Root Extract) | |
| | Prunus Amygdales Dulcis (Sweet Almond) Oil | 2.00 |
| | Dimethicone | 5.00 |
| | Homosalate | 10.00 |
| | Octocrylene | 9.40 |
| | Ethylhexyl Salicylate | 4.50 |
| | Butyl Methoxydibenzoylmethane | 3.00 |
| | Tocopheryl Acetate | 0.50 |
| B | Alcohol Denat. | to 100 |

### Water Resistant Spray SPF 30

| Phase | Raw material | w/w % |
|---|---|---|
| A | Alcohol Denat. | to 100 |
| | Polyester-10 (and) Propylene Glycol Dibenzoate | 2.00 |
| | Benzophenone-3 | 5.00 |
| | Ethylhexyl Salicylate | 5.00 |
| | Ethylhexyl Methoxycinnamate | 7.50 |
| | Homosalate | 10.00 |
| | Butyl Methoxydibenzoylmethane | 2.00 |
| | Di(iso)pentyl terephthalate | 4.00 |

### Light O/W Sun Care Lotion

| Phase | Raw material | w/w % |
|---|---|---|
| A | AXOL ® C 62 Pellets (Glyceryl Stearate Citrate) | 2.50 |
| | TEGO ® Alkanol 1618 (Cetearyl Alcohol) | 1.00 |
| | Di(iso)pentyl terephthalate | 8.00 |
| | Isoadipate (Diisopropyl Adipate) | 1.00 |
| | Butyloctyl Salicylate (HallBrite BHB, The HallStar | 2.00 |
| | Company) | |
| | Tocopheryl Acetate | 0.20 |
| | Dimethicone (5 mPas) | 1.00 |
| | Butyl Methoxydibenzoylmethane | 1.50 |
| | Diethylamino Hydroxybenzoyl Hexyl Benzoate (Uvinul | 4.00 |
| | A Plus, BASF) | |
| | Ethylhexyl Triazone | 2.00 |
| | Ethylhexyl Salicylate | 5.00 |
| | Octocrylene | 8.00 |
| | Ethylhexyl methoxycrylene | 1.50 |
| B | Titanium Dioxide; Trimethoxycaprylylsilane | 1.00 |
| C | Glycerol | 3.20 |
| | EDTA | 0.02 |
| | Water | to 100 |
| D | Acrylates/C10-30 Alkyl Acrylate Crosspolymer | 0.10 |
| | Di(iso)pentyl terephthalate | 0.90 |
| E | Sodium Hydroxide (10% in water) | q.s |
| Z | Preservative, Perfume | q.s |

### Dry Touch Hand Moisturizing Cream

| Phase | Raw material | w/w % |
|---|---|---|
| A | VARISOFT ® TA 100 (Distearyldimonium Chloride) | 3.00 |
| | Stearyl Alcohol | 1.00 |
| | TEGIN ® M Pellets (Glyceryl Stearate) | 1.00 |
| | TEGOSOFT ® SH (Stearyl Heptanoate) | 1.00 |
| | Di(iso)pentyl terephthalate | 5.00 |
| | Triacontanyl PVP | 2.00 |
| | Diethylamino Hydroxybenzoyl Hexyl Benzoate (Uvinul | 3.00 |
| | A plus, BASF) | |
| | Ethylhexyl Salicylate | 3.00 |
| | Ethylhexyl Methoxycinnamate | 5.00 |
| | Tocopheryl Acetate | 0.50 |
| | TEGO ® Feel C 10 (Cellulose) | 1.00 |
| B | Water | to 100 |
| | Glycerol | 3.00 |
| C | TEGO ® Natural Betaine (Betaine) | 2.00 |
| | Water | 2.00 |
| D | Phenoxyethanol, Ethylhexylglycerin (Euxyl PE 9010, | 0.70 |
| | Schülke & Mayr GmbH) | |

### Age Defense BB Cream SPF 15

| Phase | Raw material | w/w % |
|---|---|---|
| A | TEGIN ® 4100 Pellets (Glyceryl Stearate) | 1.00 |
| | Stearic Acid | 0.50 |
| | TEGO ® Alkanol 18, (Stearyl Alcohol) | 1.00 |
| | TEGOSOFT ® DEC (Diethylhexyl Carbonate) | 6.90 |
| | Capryloyl Glycerin/Sebacic Acid Copolymer | 3.00 |
| | HyaCare ® Filler CL (Water; Ethylhexyl Stearate; | 2.00 |
| | Sodium Hyaluronate Crosspolymer; Polyglyceryl-4 | |
| | Diisostearate/Polyhydroxystearate/Sebacate; Sodium | |
| | Isostearate) | |
| | Phytosphingosine | 0.10 |
| | Ethylhexyl Methoxycinnamate | 5.00 |
| | Diethylamino Hydroxybenzoyl Hexyl Benzoate | 3.00 |
| B | Titanium Dioxide (Hombitan AC 360, Sachtleben) | 3.00 |
| | Talc (Micro Talc IT Extra-AW, Mondo Minerals B.V.) | 2.00 |
| | Unipure Yellow LC 182 | 0.36 |
| | Unipure Red LC 381 | 0.12 |
| | Unipure Black LC 989 | 0.08 |
| | TEGOSOFT ® AC (Isoamyl Cocoate) | 4.44 |
| | Di(iso)pentyl terephthalate | 4.00 |
| | Polymethyl Methacrylate (Sepimat P, Seppic) | 3.00 |
| C | Water | to 100 |
| | Glycerol | 3.00 |
| | TEGO ® Care CG 90 (Cetearyl Glucoside) | 1.00 |
| | HyaCare ® 50 (Hydrolyzed Hyaluronic Acid) | 0.10 |
| | TEGO ® Pep 4-17 (Tetrapeptide-21; Glycerin; | 2.00 |
| | Butylene Glycol; Water) | |
| D | Alcohol | 3.00 |
| E | Microcare MEM (Dipropylene Glycol; Methylparaben; | 0.80 |
| | Ethylparaben; Water; Methylisothiazolinone) | |

### Moisture Caring BB Cream SPF 15

| Phase | Raw material | w/w % |
|---|---|---|
| A | ABIL ® EM 180 (Cetyl PEG/PPG-10/1 Dimethicone) | 2.00 |
| | ABIL ® Wax 9801 (Cetyl Dimethicone) | 2.00 |
| | Diethylamino Hydroxybenzoyl Hexyl Benzoate; | 10.00 |
| | Ethylhexyl Methoxycinnamate | |
| | Di(iso)pentyl terephthalate | 2.00 |
| | Dimethicone (and) Trimethylsiloxysilicate | 3.00 |
| | HyaCare ® Filler CL (Water; Ethylhexyl Stearate; | 2.00 |
| | Sodium Hyaluronate Crosspolymer; Polyglyceryl-4 | |
| | Diisostearate/Polyhydroxystearate/Sebacate; Sodium | |
| | Isostearate) | |
| | Phytosphingosine (Phytosphingosine) | 0.10 |
| | Hydrogenated Castor Oil | 0.50 |
| | Microcrystalline Wax | 0.50 |
| B | Talc (Micro Talc IT Extra-AW, Monde Minerals B.V.) | 2.00 |
| | Titanium Dioxide (Hombitan AC 360, Sachtleben) | 4.00 |
| | Unipure Yellow LC 182 | 0.36 |
| | Unipure Red LC 381 | 0.12 |
| | Unipure Black LC 989 | 0.08 |
| | AEROSIL R 972 (Silica Dimethylsilylate) | 0.50 |
| | Nylon-12 | 2.00 |
| | TEGOSOFT ® OS (Ethylhexyl Stearate) | 5.00 |
| | TEGOSOFT ® DEC (Diethylhexyl Carbonate) | 6.00 |
| C | Water | to 100 |
| | HyaCare ® (Sodium Hyaluronate) | 0.05 |
| | Glycerol | 5.00 |
| | Sodium Chloride | 0.80 |
| D | Phenoxyethanol; Ethylhexylglycerin (EUXYL PE 9010, | 0.70 |
| | Schülke & Mayr) | |

### Anhydrous Stick

| Phase | Raw material | w/w % |
|---|---|---|
| A | dermofeel ® viscolid MB (Hydrogenated Vegetable Oil) | 10.00 |
| | Poly C10-30 Alkyl Acrylate | 10.00 |
| | Di(iso)pentyl terephthalate | 30.00 |
| | Isopropyl Palmitate | 15.00 |
| | Octinoxate (Ethylhexyl Methoxycinnamate) | 7.50 |
| | Octyldodecanol | to 100 |

### O/W Sun Protect & Bronze

| Phase | Raw material | w/w % |
|---|---|---|
| A | AXOL ® C 62 Pellets (Glyceryl Stearate Citrate) | 2.00 |
| | TEGO ® Alkanol 1618 (Cetearyl Alcohol) | 1.50 |
| | Di(iso)pentyl terephthalate | 8.00 |
| | C12-15 Alkyl Benzoate | 4.00 |
| | Cera Alba (Beeswax 8104, KahlWax) | 1.00 |
| | Titanium Dioxide, Trimethoxycaprylylsilane | 1.50 |
| | Dimethicone (5 cSt) | 3.00 |
| | Octocrylene | 9.00 |
| | Ethylhexyl Salicylate | 4.50 |
| | Butyl Methoxydibenzoylmethane | 2.00 |
| | Tocopheryl Acetate | 0.50 |
| | Xanthan Gum | 0.50 |
| B | Water | to 100 |
| | Glycerol | 3.00 |
| | Panthenol | 1.00 |
| C | Acrylates/C10-30 Alkyl Acrylate Crosspolymer | 0.10 |
| | C12-15 Alkyl Benzoate | 0.40 |
| D | Sodium Hydroxide (10% in water) | q.s. |
| E | Butylene Glycol; Acetyl-L-Tyrosine; Hydrolyzed | 0.80 |
| | Vegetable Protein; Adenosine Triphosphate; | |
| | Riboflavin (Unipertan VEG-2001) | |
| Z | Preservative, Perfume | q.s. |

### Sun Care Foam SPF 50

| Phase | Raw material | w/w % |
|---|---|---|
| A | TEGO ® Care PBS 6 (Polyglyceryl-6 Stearate (and) | 3.00 |
| | Polyglyceryl-6 Behenate) | |
| | Di(iso)pentyl terephthalate | 5.00 |
| | dermofeel ® viscolid MB (Hydrogenated Vegetable Oil) | 0.50 |
| | Bis-Ethylhexyloxyphenol Methoxyphenyl Triazine | 4.50 |
| | Butyl Methoxydibenzoylmethane | 3.00 |
| | Octocrylene | 8.00 |
| | Homomenthyl Salicylate | 6.00 |
| | Ethylhexyl Salicylate | 2.50 |
| | TEGO ® Feel C 10 (Cellulose) | 2.00 |
| B | Water | to 100 |
| | Glycerol | 2.00 |
| | EDTA Disodium | 0.05 |
| C | C12-15 Alkyl Benzoate | 0.80 |
| | Carbomer | 0.20 |
| | Xanthan Gum (Keltrol CG-SFT, CP Kelco) | 0.20 |
| D | Water | 11.68 |
| | Phenylbenzimidazole Sulfonic Acid | 3.00 |
| | Tromethamine | 1.32 |
| E | Phenoxyethanol, Ethylhexylglycerin (Euxyl PE 9010, | 0.70 |
| | Schülke & Mayr GmbH) | |
| | TEGO ® Betain 810 (Capryl/Capramidopropyl Betaine) | 1.00 |
| | REWOTERIC ® AM C (Sodium Cocoamphoacetate) | 1.00 |
| F | Tromethamine (30% in water) | q.s. |

### W/O Sun Protection Shake-Shake

| Phase | Raw material | w/w % |
|---|---|---|
| A | ABIL ® EM 90 (Cetyl PEG/PPG-10/1 Dimethicone) | 1.00 |
| | Di(iso)pentyl terephthalate | 11.00 |
| | TEGOSOFT ® DEC (Diethylhexyl Carbonate) | 2.50 |
| | Dimethicone (5 cSt) | 12.00 |
| | Octocrylene | 6.00 |
| | Ethylhexyl Salicylate | 2.00 |
| | Butyl Methoxydibenzoylmethane | 0.80 |
| | Bis-Ethylhexyloxyphenol Methoxyphenyl Triazine | 1.50 |
| | (Tinosorb S, BASF SE) | |
| | Titanium Dioxide, Trimethoxycaprylylsilane | 2.00 |
| | VP/Hexadecene Copolymer (Antaron V-216, Ashland) | 0.50 |
| B | Water | 4.43 |
| | Phenylbenzimidazole Sulfonic Acid (Eusolex 232, | 0.50 |
| | Merck KGaA) | |
| | Sodium Hydroxide | 0.07 |
| C | Distarch Phosphate (MAIS PO4 PH “B”, Agrana Stärke | 1.50 |
| | GmbH) | |
| | Alcohol | 1.00 |
| | Glycerol | 6.00 |
| | Water | to 100 |
| | Preservative, Perfume | q.s. |

### Light Sun Care w/o Shake-Shake SPF30 PA+++

| Phase | Raw material | w/w % |
|---|---|---|
| A | ABIL ® EM 90 (Cetyl PEG/PPG-10/1 Dimethicone) | 1.50 |
| | TEGOSOFT ® DEC (Diethylhexyl Carbonate) | 5.00 |
| | Dimethicone (2 cSt) | 5.00 |
| | Cyclopentasiloxane | 15.00 |
| | Di(iso)pentyl terephthalate | 5.00 |
| | C12-15 Alkyl Benzoate; Titanium Dioxide; PEG-30 | 2.40 |
| | Dipolyhydroxystearate; Silica; Dimethicone(T-lique | |
| | AB50Si, UNI-POWDER) | |
| | Zinc Oxide(ZnO-NAS, SUNJIN) | 8.00 |
| | ABIL ® Wax 9801 (Cetyl Dimethicone) | 2.00 |
| | Talc | 2.00 |
| | Polymethylsilsesquioxane (Gransil PSQ, Grant) | 4.00 |
| | Vinyl Dimethicone/Methicone Silsesquioxane | 2.00 |
| | Crosspolymer (KSP-100, ShinEtsu) | |
| | Diethylamino Hydroxybenzoyl Hexyl Benzoate | 2.00 |
| | Ethylhexyl Methoxycinnamate | 8.00 |
| | Ethylhexyl Salicylate | 4.00 |
| | Bis-Ethylhexyloxyphenol Methoxyphenyl Triazine | 0.50 |
| | (Escalol S, Ashland) | |
| B | Water | to 100 |
| | Glycerol | 3.00 |
| | Sodium Chloride | 1.00 |
| C | Phenoxyethanol, Ethylhexylglycerin (Euxyl PE 9010, | 0.70 |
| | Schülke & Mayr GmbH) | |

### W/O Organic Shake-Shake SPF50+ PA++++

| Phase | Raw material | w/w % |
|---|---|---|
| A | ABIL ® EM 90 (Cetyl PEG/PPG-10/1 Dimethicone) | 1.50 |
| | TEGOSOFT ® DEC (Diethylhexyl Carbonate) | 7.00 |
| | Dimethicone (5 cSt) | 5.00 |
| | Cyclopentasiloxane | 5.00 |
| | Di(iso)pentyl terephthalate | 3.00 |
| | C12-15 Alkyl Benzoate; Titanium Dioxide; PEG-30 | 15.60 |
| | Dipolyhydroxystearate; Silica; Dimethicone(T-lique | |
| | AB50Si, UNI-POWDER) | |
| | ABIL ® Wax 9801 (Cetyl Dimethicone) | 2.00 |
| | Bis-Ethylhexyloxyphenol Methoxyphenyl Triazine | 1.00 |
| | (Escalol S, Ashland) | |
| | Diethylamino Hydroxybenzoyl Hexyl Benzoate (Uvinul | 1.50 |
| | A plus Granular, BASF) | |
| | Octocrylene | 2.00 |
| | Ethylhexyl Methoxycinnamate | 7.00 |
| | Ethylhexyl Salicylate | 4.00 |
| | Zinc Oxide(ZnO-NAS, SUNJIN) | 14.40 |
| B | Water | to 100 |
| | Butylene Glycol | 3.00 |
| | Sodium Chloride | 1.50 |
| C | Phenoxyethanol, Ethylhexylglycerin (Euxyl PE 9010, | 0.70 |
| | Schülke & Mayr GmbH) | |

### Summer Paradise Cream SPF 30

| Phase | Raw material | w/w % |
|---|---|---|
| A | TEGO ® Care PBS 6 MB (Polyglyceryl-6 Stearate, | 3.00 |
| | Polyglyceryl-6 Behenate) | |
| | TEGIN ® M Pellets MB (Glyceryl Stearate) | 0.50 |
| | TEGO ® Alkanol 1618 (Cetearyl Alcohol) | 0.50 |
| | Di(iso)pentyl terephthalate | 4.50 |
| | Bis-Ethylhexyloxyphenol Methoxyphenyl Triazine | 3.00 |
| | (Tinosorb S, BASF) | |
| | Butyl Methoxydibenzoylmethane | 2.00 |
| | Ethylhexyl Salicylate | 3.00 |
| | Octocrylene | 8.00 |
| | Homosalate | 3.00 |
| | Ethylhexyl Triazone | 1.50 |
| | TEGO ® SP 13-1 (Poly C10-30 Alkyl Acrylate) | 1.50 |
| B | Water | to 100 |
| | Glycerol | 3.00 |
| | EDTA | 0.05 |
| | TEGO ® Feel C 10 (Cellulose) | 2.00 |
| C | Acrylates/C10-30 Alkyl Acrylate Crosspolymer | 0.20 |
| | Di(iso)pentyl terephthalate | 0.80 |
| D | Sodium Hydroxide (10% in water) | q.s. |
| E | Verstatil ® PC (Phenoxyethanol, Caprylyl Glycol) | 1.00 |

### On the Go UV Protection Stick SPF 50

| Phase | Raw material | w/w % |
|---|---|---|
| A | Di(iso)pentyl terephthalate | 8.00 |
| | TEGOSOFT ® CT (Caprylic/Capric Triglyceride) | 7.50 |
| | TEGOSOFT ® DEC (Diethylhexyl Carbonate) | 8.60 |
| | Titanium Dioxide, Silica, Dimethicone (Parsol TX, | 2.50 |
| | DSM) | |
| B | TEGOSOFT ® CR MB (Cetyl Ricinoleate) | 3.00 |
| | TEGOSOFT ® MM MB (Myristyl Myristate) | 5.00 |
| | Diethylamino Hydroxybenzoyl Hexyl Benzoate | 8.00 |
| | Ethylhexyl Salicylate | 5.00 |
| | Ethylhexyl Methoxycinnamate | 10.00 |
| | Homosalate | 5.00 |
| | Bis-Ethylhexyloxyphenol Methoxyphenyl Triazine | 4.00 |
| | (Tinosorb S, BASF) | |
| | Beeswax | 4.00 |
| | Polyglyceryl-3 Beeswax (Cera Bellina, Koster Keunen) | 6.00 |
| | Ozokerite (1899, KahlWax) | 3.50 |
| | Copernicia Cerifera (Carnauba) Wax (2442L, KahlWax) | 2.50 |
| | dermofeel ® Ascorbyl Palmitate (Ascorbyl Palmitate) | 0.10 |
| | dermofeel ® Toco 70 non GMO (Tocopherol, | 0.20 |
| | Helianthus Annuus (Sunflower) Seed Oil) | |
| | TEGO ® SP 13-1 (Poly C10-30 Alkyl Acrylate) | 5.00 |
| C | AEROSIL ® 200 (Silica) | 2.00 |
| D | TEGO ® Feel C 10 (Cellulose) | 10.00 |
| Z | Perfume | q.s. |

### Transparent UV Protection Water Spray SPF 30

| Phase | Raw material | w/w % |
|---|---|---|
| A | TEGOSOFT ® DEC (Diethylhexyl Carbonate) | 15.00 |
| | Isododecane | 7.50 |
| | Di(iso)pentyl terephthalate | 5.00 |
| | dermofeel ® sensolv MB (Isoamyl Laurate) | 5.00 |
| | Butyl Methoxydibenzoylmethane | 5.00 |
| | Bis-Ethylhexyloxyphenol Methoxyphenyl Triazine | 2.40 |
| | (Tinosorb S, BASF) | |
| | Homosalate | 2.00 |
| | Ethylhexyl Salicylate | 2.00 |
| | Ethylhexyl Triazone | 1.70 |
| | dermofeel ® Toco 70 non GMO (Tocopherol, | 0.50 |
| | Helianthus Annuus (Sunflower) Seed Oil) | |
| | TEGO ® SP 13-1 (Poly C10-30 Alkyl Acrylate) | 0.35 |
| B | Verstatil ® PC (Phenoxyethanol, Caprylyl Glycol) | 1.00 |
| | Perfume | q.s. |
| C | Water | to 100 |
| | Glycerol | 3.00 |
| | Disodium EDTA | 0.10 |
| D | Water | 9.65 |
| | Phenylbenzimidazole Sulfonic Acid | 2.50 |
| | Sodium Hydroxide | 0.35 |
| E | Triethanolamine¹⁾ | q.s. |

### Sun Care Cream SPF 25

| Phase | Raw material | w/w % |
|---|---|---|
| A | TEGO ® Care PBS 6 MB (Polyglyceryl-6 Stearate, | 3.00 |
| | Polyglyceryl-6 Behenate) | |
| | TEGIN ® M Pellets MB (Glyceryl Stearate) | 0.50 |
| | TEGO ® Alkanol 1618 (Cetearyl Alcohol) | 0.50 |
| | Di(iso)pentyl terephthalate | 3.50 |
| | Butyl Methoxydibenzoylmethane | 3.00 |
| | Ethylhexyl Salicylate | 5.00 |
| | Octocrylene | 10.0 |
| | Homosalate | 10.0 |
| | VP/Eicosene Copolymer | 1.50 |
| B | Water | to 100 |
| | Glycerol | 3.00 |
| | EDTA | 0.05 |
| C | Acrylates/C10-30 Alkyl Acrylate Crosspolymer | 0.20 |
| | Di(iso)pentyl terephthalate | 0.80 |
| D | Sodium Hydroxide (10% in water) | 0.60 |
| E | Verstatil ® PC (Phenoxyethanol, Caprylyl Glycol) | 1.00 |

### Inorganic Water Resistant O/W Sunscreen SPF 20

| Phase | Raw material | w/w % |
|---|---|---|
| A | TEGO ® Care PBS 6 (Polyglyceryl-6 Stearate; | 4.00 |
| | Polyglyceryl-6 Behenate) | |
| | TEGO ® SP 13-1 (Poly C10-30 Alkyl Acrylate) | 1.50 |
| | TEGIN ® M Pellets MB (Glyceryl Stearate) | 0.20 |
| | TEGO ® Alkanol 1618 (Cetearyl Alcohol) | 0.20 |
| B | Zinc Oxide; Triethoxycaprylylsilane (Z-Cote HP-1, | 7.80 |
| | BASF) | |
| | Titanium Dioxide, Silica, Dimethicone (Parsol TX, | 7.20 |
| | DSM) | |
| | Di(iso)pentyl terephthalate | 3.00 |
| | dermofeel ® sensolv MB (Isoamyl Laurate) | 3.00 |
| | TEGOSOFT ® CT (Caprylic/Capric Triglyceride) | 5.00 |
| | TEGOSOFT ® DEC (Diethylhexyl Carbonate) | 5.00 |
| | ABIL ® 350 (Dimethicone) | 3.00 |
| | ABIL ® Wax 9840 (Cetyl Dimethicone) | 2.00 |
| | dermosoft ® Octiol (Caprylyl Glycol) | 0.50 |
| C | Water | to 100 |
| | Glycerol | 3.00 |
| | Xanthan Gum | 0.50 |
| D | dermosoft ® Pentiol eco (Pentylene Glycol) | 3.00 |
| E | Citric acid (50% in water) | 0.45 |

### Feel the Sun Spray SPF 50

| Phase | Raw material | w/w % |
|---|---|---|
| A | TEGO ® Care PBS 6 MB (Polyglyceryl-6 Stearate, | 4.00 |
| | Polyglyceryl-6 Behenate) | |
| | TEGIN ® M Pellets MB (Glyceryl Stearate) | 0.25 |
| | TEGO ® Alkanol 1618 (Cetearyl Alcohol) | 0.25 |
| | dermofeel ® sensolv MB (Isoamyl Laurate) | 4.00 |
| | Di(iso)pentyl terephthalate | 4.00 |
| | Bis-Ethylhexyloxyphenol Methoxyphenyl Triazine | 6.00 |
| | (Tinosorb S, BASF) | |
| | Diethylamino Hydroxybenzoyl Hexyl Benzoate | 6.00 |
| | Ethylhexyl Salicylate | 5.00 |
| | Ethylhexyl Triazone | 2.00 |
| | dermofeel ® Toco 70 non GMO | 0.20 |
| | (Tocopherol, Helianthus Annuus (Sunflower) Seed Oil) | |
| B | Water | to 100 |
| | Glycerol | 3.00 |
| | EDTA | 0.05 |
| C | Xanthan Gum (Keltrol CG-SFT, CP Kelco) | 0.50 |
| D | Phenylbenzimidazole Sulfonic Acid | 3.00 |
| | Tromethamine (Trisaminomethane) | 1.32 |
| | Water | 10.68 |
| E | Verstatil ® BOB (Benzyl Alcohol; Caprylyl Glycol; | 1.20 |
| | Benzoic Acid) | |
| | Perfume | 0.10 |

### Sun Protection Stick

| Phase | Raw material | w/w % |
|---|---|---|
| A | Carthamus Tinctorius (Safflower) Seed Oil | 22.80 |
| | Butyrospermum Parkii (Shea) Butter | 7.00 |
| | Hippophae Rhamnoides Fruit Oil | 1.00 |
| | dermofeel ® sensolv MB (Isoamyl Laurate) | 8.00 |
| | Di(iso)pentyl terephthalate | 2.00 |
| | dermofeel ® viscolid MB (Hydrogenated Vegetable Oil) | 10.00 |
| | TEGOSOFT ® DC MB (Decyl Cocoate) | 6.00 |
| | TEGOSOFT ® OER MB (Oleyl Erucate) | 6.00 |
| | Cera Alba (Beeswax 8104, KahlWax) | 5.00 |
| | Helianthus Annuus Seed Cera, Ascorbyl Palmitate, | 3.00 |
| | Tocopherol (Sunflower Seed Wax 6607L, KahlWax) | |
| | Euphorbia Cerifera Cera (Candelilla Wax 2039L, | 3.00 |
| | KahlWax) | |
| | dermofeel ® TocoBalance (Tocopherol, | 1.50 |
| | Helianthus Annuus (Sunflower) Seed Oil) | |
| | dermosoft ® GMCY MB (Glyceryl Caprylate) | 1.00 |
| B | Distarch Phosphate (Corn P04 PH “B”, Agrana) | 1.00 |
| | TEGO ® Feel C 10 (Cellulose) | 2.00 |
| | Zinc Oxide (Zano 10, EverZinc) | 20.00 |
| C | Perfume | 0.70 |

### W/O Quick-Breaking Cream SPF 15

| Phase | Raw material | w/w % |
|---|---|---|
| A | ABIL ® EM 180 (Cetyl PEG/PPG-10/1 Dimethicone) | 0.80 |
| | TEGOSOFT ® DEC (Diethylhexyl Carbonate) | 3.00 |
| | Di(iso)pentyl terephthalate | 2.00 |
| | Dimethicone (5 cSt) | 3.00 |
| | TEGO ® SP 13-6 (Poly C10-30 Alkyl Acrylate) | 1.00 |
| | Bis-ethylhexyloxyphenol Methoxyphenyl Triazine | 2.00 |
| | (Tinosorb S, BASF) | |
| | Diethylamino Hydroxybenzoyl Hexyl Benzoate | 2.00 |
| | 4-Methylbenzylidene Camphor | 4.00 |
| | Ethylhexyl Salicylate | 2.00 |
| | Verstatil ® BOB (Benzyl Alcohol, Caprylyl Glycol, | 1.00 |
| | Benzoic Acid) | |
| B | Water | to 100 |
| | Propylene Glycol | 5.00 |
| | Sodium Chloride | 1.00 |

### Fun in the Sun SPF 30 Spray

| Phase | Raw material | w/w % |
|---|---|---|
| A | TEGO ® Care PBS 6 MB (Polyglyceryl-6 Stearate, | 3.00 |
| | Polyglyceryl-6 Behenate) | |
| | Di(iso)pentyl terephthalate | 3.00 |
| | Bis-ethylhexyloxyphenol Methoxyphenyl Triazine | 3.00 |
| | (Tinosorb S, BASF) | |
| | Butyl Methoxydibenzoylmethane | 2.00 |
| | Homosalate | 4.00 |
| | Ethylhexyl Salicylate | 4.00 |
| | Octocrylene | 4.00 |
| | TEGO ® Feel C 10 (Cellulose) | 1.00 |
| B | Water | to 100 |
| | Gellan Gum (Kelcogel CG-HA, CP Kelco) | 0.03 |
| | EDTA | 0.05 |
| | Glycerol | 3.00 |
| | Acrylates/C10-30 Alkyl Acrylate Crosspolymer | 0.20 |
| C | Phenylbenzimidazole Sulfonic Acid | 2.00 |
| | Tromethamine | 0.88 |
| | Water | 7.12 |
| E | Tromethamine (Trisaminomethane, 30% in water) | q.s. |
| F | dermofeel ® OMP (Methylpropanediol, Caprylyl Glycol, | 3.00 |
| | Phenylpropanol) | |

### Cationic Sun Screen SPF 12

| Phase | Raw material | w/w % |
|---|---|---|
| A | VARISOFT ® TA 100 (Distearyldimonium Chloride) | 3.50 |
| | TEGIN ® M Pellets (Glyceryl Stearate) | 1.50 |
| | TEGO ® Alkanol 18 (Stearyl Alcohol) | 1.00 |
| | Di(iso)pentyl terephthalate | 5.00 |
| | TEGOSOFT ® DEC (Diethylhexyl Carbonate) | 3.00 |
| | TEGOSOFT ® CR (Cetyl Ricinoleate) | 1.00 |
| | Triisostearin | 1.00 |
| | Ethylhexyl Methoxycinnamate | 5.00 |
| | Ethylhexyl Salicylate | 4.00 |
| | Menthyl Anthranilate | 4.00 |
| B | TEGO ® Cosmo C 100 (Creatine) | 0.50 |
| | Glycerol | 3.00 |
| | Water | to 100 |
| Z | Preservative, perfume | q.s. |

### W/O Sun Care Lotion SPF 8, Water Resistant

| Phase | Raw material | w/w % |
|---|---|---|
| A | ISOLAN ® GPS (Polyglyceryl-4 Diisostearate/ | 3.00 |
| | Polyhydroxystearate/Sebacate) | |
| | Hydrogenated Castor Oil | 0.20 |
| | Microcrystalline Wax | 0.30 |
| | Di(iso)pentyl terephthalate | 5.00 |
| | TEGOSOFT ® DEC (Diethylhexyl Carbonate) | 3.00 |
| | TEGOSOFT ® CR (Cetyl Ricinoleate) | 1.00 |
| | Triisostearin | 1.00 |
| | Ethylhexyl Methoxycinnamate | 5.00 |
| | Ethylhexyl Salicylate | 4.00 |
| | Menthyl Anthranilate | 4.00 |
| B | TEGO ® Cosmo C 100 (Creatine) | 0.50 |
| | Glycerol | 3.00 |
| | Magnesium Sulfate Heptahydrate | 1.50 |
| | Water | to 100 |
| Z | Preservative, perfume | q.s. |

### Cationic Sun Screen SPF 19, Water Resistant

| Phase | Raw material | w/w % |
|---|---|---|
| A | VARISOFT ® TA 100 (Distearyldimonium Chloride) | 3.50 |
| | TEGIN ® M Pellets (Glyceryl Stearate) | 2.00 |
| | TEGO ® Alkanol 18 (Stearyl Alcohol) | 1.00 |
| | Di(iso)pentyl terephthalate | 5.00 |
| | TEGOSOFT ® DEC (Diethylhexyl Carbonate) | 3.50 |
| | TEGOSOFT ® CR (Cetyl Ricinoleate) | 1.00 |
| | Triisostearin | 1.00 |
| | Titanium Dioxide, Diethylhexyl Carbonate, | 5.00 |
| | Polyglyceryl-6 Polyhydroxystearate | |
| | 4-Methylbenzylidene Camphor | 4.00 |
| | Octocrylene | 3.00 |
| | Butyl Methoxydibenzoylmethane | 2.00 |
| B | Glycerol | 3.00 |
| | Water | to 100 |
| Z | Preservative, perfume | q.s. |

### Cationic Sun Screen SPF 15, Water Resistant

| Phase | Raw material | w/w % |
|---|---|---|
| A | VARISOFT ® TA 100 (Distearyldimonium Chloride) | 3.50 |
| | TEGIN ® M Pellets (Glyceryl Stearate) | 1.50 |
| | TEGO ® Alkanol 18 (Stearyl Alcohol) | 1.00 |
| | Di(iso)pentyl terephthalate | 8.00 |
| | TEGOSOFT ® DEC (Diethylhexyl Carbonate) | 5.00 |
| | TEGOSOFT ® CR (Cetyl Ricinoleate) | 1.00 |
| | Triisostearin | 1.00 |
| | Ethylhexyl Methoxycinnamate | 5.00 |
| | Bis-Ethylhexyloxyphenol Methoxyphenyl Triazine | 3.00 |
| B | TEGO ® Cosmo C 100 (Creatine) | 0.50 |
| | Glycerol | 3.00 |
| | Water | to 100 |
| Z | Preservative, perfume | q.s. |

### Cationic Sun Care Cream SPF 14

| Phase | Raw material | w/w % |
|---|---|---|
| A | VARISOFT ® PATC (Palmitamidopropyltrimonium | 1.20 |
| | Chloride) | |
| | TEGIN ® M Pellets (Glyceryl Stearate) | 1.00 |
| | TEGO ® Alkanol 1618 (Cetearyl Alcohol) | 2.80 |
| | Di(iso)pentyl terephthalate | 2.60 |
| | TEGOSOFT ® DEC (Diethylhexyl Carbonate) | 3.00 |
| | Titanium Dioxide, Diethylhexyl Carbonate, | 4.40 |
| | Polyglyceryl-6 Polyhydroxystearate | |
| | Octocrylene | 5.00 |
| | Ethylhexyl Methoxycinnamate | 5.00 |
| | Ethylhexyl Salicylate | 3.00 |
| | Butyl Methoxydibenzoylmethane | 2.00 |
| B | Glycerol | 3.00 |
| | Water | to 100 |
| Z | Preservative, perfume | q. s. |

### Everyday Sunshine Cream SPF 15

| Phase | Raw material | w/w % |
|---|---|---|
| A | TEGO ® Care 165 (Glyceryl Stearate; PEG-100 | 3.00 |
| | Stearate) | |
| | TEGO ® Alkanol 18, (Stearyl Alcohol) | 2.00 |
| | Di(iso)pentyl terephthalate | 2.00 |
| | Propylene Glycol Dibenzoate | 2.00 |
| | TEGOSOFT ® CT (Caprylic/Capric Triglyceride) | 2.00 |
| | Titanium Dioxide; Trimethoxycaprylylsilane | 1.00 |
| | Paraffinum Liquidum | 2.00 |
| | Butyrospermum Parkii (Shea Butter) | 1.00 |
| | Octocrylene | 2.00 |
| | Butyl Methoxydibenzoylmethane | 2.00 |
| | Ethylhexyl Salicylate | 5.00 |
| | Tocopheryl Acetate | 0.80 |
| B | Water | to 100 |
| | Glycerol | 3.00 |
| | Graham's salt (Sodium Hexametaphosphate) | 0.20 |
| | TEGO ® Cosmo C 100 (Creatine) | 0.50 |
| | HyaCare ® 50 (Hydrolyzed Hyaluronic Acid) | 0.10 |
| | Polyacrylamide; C13-14 Isoparaffin; Laureth-7 | 2.00 |
| | (Sepigel 305, Seppic) | |
| D | Water | 13.29 |
| | Phenylbenzimidazole Sulfonic Acid (Eusolex 232, | 1.05 |
| | Merck KGaA) | |
| | Sodium Hydroxide | 0.21 |
| Z | Preservative, perfume | q.s. |

### High Sun Protection Lotion O/W SPF 50

| Phase | Raw material | w/w % |
|---|---|---|
| A | ABIL ® Wax 9840 (Cetyl Dimethicone) | 0.50 |
| | Di(iso)pentyl terephthalate | 5.00 |
| | REWOPAL ® PIB 1000 (Polyisobutene) | 0.50 |
| | Acrylates/C10-30 Alkyl Acrylate Crosspolymer | 0.20 |
| | Xanthan Gum | 0.20 |
| | Titanium Dioxide; Trimethoxycaprylylsilane | 1.50 |
| | Bis-Ethylhexyloxyphenol Methoxyphenyl Triazine | 4.00 |
| | (Tinosorb S, BASF SE) | |
| | Octocrylene | 10.00 |
| | Butyl Methoxydibenzoylmethane | 4.00 |
| B | TEGO ® Care CG 90 (Cetearyl Glucoside) | 0.50 |
| | Glycerol | 4.00 |
| | Water | to 100 |
| C | Alcohol | 5.00 |
| | Phenoxyethanol, Methylparaben, Propylparaben, | 1.00 |
| | Ethylparaben (Phenonip XB, Clariant) | |
| D | Methylene Bis-Benzotriazolyl Tetramethylbutylphenol** | 8.00 |
| | (Tinosorb M, BASF SE) | |
| E | Sodium Hydroxide (10% in water) | q.s. |

### Icy O/W Sun Care Lotion SPF 25

| Phase | Raw material | w/w % |
|---|---|---|
| A | TEGO ® Care PS (Methyl Glucose Sesquistearate) | 2.00 |
| | TEGO ® Alkanol 18, (Stearyl Alcohol) | 1.00 |
| | Di(iso)pentyl terephthalate | 5.00 |
| | TEGOSOFT ® SH (Stearyl Heptanoate) | 3.00 |
| | TEGO ® Xymenynic (Caprylic/Capric Triglyceride, | 1.00 |
| | Xymenynic Acid) | |
| | Diethylamino Hydroxybenzoyl Hexyl Benzoate | 6.00 |
| | 4-Methylbenzylidene Camphor | 4.00 |
| | Diethylhexyl Butamido Triazone | 3.00 |
| | Xanthan Gum | 0.50 |
| B | Water | to 100 |
| | Glycerol | 3.00 |
| C | Carbomer | 0.10 |
| | TEGOSOFT ® XC (Phenoxyethyl Caprylate) | 0.40 |
| D | Sodium Hydroxide (10% in water) | q.s. |
| E | Dipropylene Glycol; Methylparaben; Ethylparaben; | 0.80 |
| | Water; Methylisothiazolinone (Microcare MEM, Thor) | |
| Z | Perfume | q.s. |

### Low Viscosity W/O Suncare Lotion SPF30 PA+++

| Phase | Raw material | w/w % |
|---|---|---|
| A | ABIL ® EM 90 (Cetyl PEG/PPG-10/1 Dimethicone) | 2.50 |
| | TEGOSOFT ® DEC (Diethylhexyl Carbonate) | 2.00 |
| | Di(iso)pentyl terephthalate | 2.00 |
| | Dimethicone (DC200, 5 cst, Dow Corning) | 16.00 |
| | Titanium Dioxide (Parsol TX, DSM) | 10.00 |
| | Zinc Oxide (SUNCLEAR-Z60CO, SUNJIN) | 25.00 |
| | Dimethicone/Vinyl Dimethicone Crosspolymer and Silica | 1.00 |
| | (DC 9701, DowCorning) | |
| B | Water | to 100 |
| | Butylene Glycol | 5.00 |
| | Sodium Chloride | 1.50 |
| C | Phenoxyethanol, Ethylhexylglycerin (Euxyl PE 9010, | 0.70 |
| | Schülke & Mayr GmbH) | |

### O/W Sun Care Gel SPF30 PA+++

| Phase | Raw material | w/w % |
|---|---|---|
| A | ABIL ® Care XL 80 (Bis-PEG/PPG-20/5 PEG/PPG-20/5 | 1.00 |
| | Dimethicone; Methoxy PEG/PPG-25/4 Dimethicone; | |
| | Caprylic/Capric Triglyceride) | |
| | TEGOSOFT ® DEC (Diethylhexyl Carbonate) | 4.00 |
| | Dimethicone (2 cSt) | 3.00 |
| | Di(iso)pentyl terephthalate | 2.00 |
| | ABIL ® Wax 9801 (Cetyl Dimethicone) | 2.00 |
| | Bis-ethylhexyloxyphenol methoxyphenyl triazine | 3.00 |
| | (Tinosorb S, BASF) | |
| | Diethylamino Hydroxybenzoyl Hexyl Benzoate (Uvinul | 4.00 |
| | A plus Granular, BASF) | |
| | Octocrylene | 2.00 |
| | Ethylhexyl Methoxycinnamate | 8.00 |
| B | Water | to 100 |
| | Butylene Glycol | 5.00 |
| | Xanthan Gum | 0.20 |
| C | Sodium Acrylate/Sodium Acryloyldimethyl Taurate | 2.00 |
| | Copolymer (and) Isohexadecane (and) Polysorbate-80 | |
| | (Simulgel EG, Seppic) | |
| D | Phenoxyethanol, Ethylhexylglycerin (Euxyl PE 9010, | 0.70 |
| | Schülke & Mayr GmbH) | |

### Sun Care Aqua Gel SPF50+, PA++++

| Phase | Raw material | w/w % |
|---|---|---|
| A | ABIL ® Care XL80 (Bis-PEG/PPG-20/5 PEG/PPG-20/5 | 3.00 |
| | Dimethicone; Methoxy PEG/PPG-25/4 Dimethicone; | |
| | Caprylic/Capric Triglyceride) | |
| | TEGOSOFT ® DEC (Diethylhexyl Carbonate) | 7.00 |
| | Dimethicone (5 cSt) | 2.00 |
| | Di(iso)pentyl terephthalate | 3.00 |
| | ABIL ® Wax 9801 (Cetyl Dimethicone) | 2.00 |
| | Bis-ethylhexyloxyphenol methoxyphenyl triazine | 3.00 |
| | (Tinosorb S, BASF) | |
| | Diethylamino Hydroxybenzoyl Hexyl Benzoate (Uvinul | 4.00 |
| | A plus Granular, BASF) | |
| | Octocrylene | 5.00 |
| | Avobenzone(Parsol 1789, DSM) | 3.00 |
| | Homomenthyl Salicylate | 8.00 |
| | Ethylhexyl Salicylate | 5.00 |
| | Ethylhexyl Methoxycinnamate | 10.00 |
| B | Water | to 100 |
| | Butylene Glycol | 3.00 |
| | Xanthan Gum | 0.20 |
| C | Sodium Acrylate/Sodium Acryloyldimethyl Taurate | 1.50 |
| | Copolymer (and) Isohexadecane (and) Polysorbate-80 | |
| | (Simulgel EG, SEPPIC) | |
| D | Phenoxyethanol, Ethylhexylglycerin (Euxyl PE 9010, | 0.70 |
| | Schülke & Mayr GmbH) | |

### O/W Sun Care Cream with High Protection SPF 50+ PA+++

| Phase | Raw material | w/w % |
|---|---|---|
| A | TEGO ® Care 450 (Polyglyceryl-3 Methylglucose | 3.00 |
| | Distearate) | |
| | Di(iso)pentyl terephthalate | 2.00 |
| | TEGO ® Alkanol 1618 (Cetearyl Alcohol) | 1.00 |
| | ABIL ® Wax 9840 (Cetyl Dimethicone) | 0.50 |
| | Bis-ethylhexyloxyphenol Methoxyphenyl Triazine | 2.00 |
| | (Tinosorb S, BASF SE) | |
| | Butyl Methoxydibenzoylmethane | 3.00 |
| | Octocrylene | 8.00 |
| | Ethylhexyl Salicylate | 5.00 |
| | C12-15 Alkyl Benzoate; Titanium Dioxide; PEG-30 | 8.90 |
| | Dipolyhydroxystearate; Silica; Dimethicone | |
| | (T-lique AB50Si, UNI-POWDER) | |
| B | Water | to 100 |
| | Glycerol | 3.00 |
| C | Phenylbenzimidazole Sulfonic Acid | 3.00 |
| | Tromethamine 78% | 2.00 |
| | Water | 4.00 |
| D | Acrylates/C10-30 Alkyl Acrylate Crosspolymer (2% | 10.00 |
| | solution in water) | |
| E | Sodium Hydroxide (10% in water) | 0.40 |
| F | Phenoxyethanol, Ethylhexylglycerin (Euxyl PE 9010, | 0.70 |
| | Schülke & Mayr GmbH) | |

### Transparent Sun Stick SPF50+, PA++++

| Phase | Raw material | w/w % |
|---|---|---|
| A | Octyldodecanol | 24.00 |
| | Dibutyl Ethyhexanoyl Glutamide (EB-21) | 3.00 |
| | Dibutyl Lauroyl Glutamide (GP-1) | 3.00 |
| B | TEGOSOFT ® Liquid (Cetearyl Ethylhexanoate) | 10.00 |
| | TEGOSOFT ® OP (Ethylhexyl Palmitate) | 15.00 |
| | Di(iso)pentyl terephthalate | 1.50 |
| | ABIL ® Wax 9801 (Cetyl Dimethicone) | 1.00 |
| | REWOPAL ® PIB 1000 (Polyisobutene) | 6.00 |
| | AEROSIL R 812S (silica) | 7.00 |
| C | Ethylhexyl Methoxycinnamate | 7.00 |
| | Ethylhexyl Salicylate | 5.00 |
| | Octocrylene | 7.00 |
| | Bis-ethylhexyloxyphenol Methoxyphenyl Triazine | 5.50 |
| | (Tinosorb S, BASF SE) | |
| | Diethylamino Hydroxybenzoyl Hexyl Benzoate (Uvinul | 5.00 |
| | A plus Granular, BASF) | |

### Sun Care Cream SPF 15

| Phase | Raw material | w/w % |
|---|---|---|
| A | TEGO ® Care 450 (Polyglyceryl-3 Methylglucose | 3.00 |
| | Distearate) | |
| | TEGIN ® M Pellets (Glyceryl Stearate) | 1.50 |
| | TEGO ® Alkanol 1618 (Cetearyl Alcohol) | 1.50 |
| | Di(iso)pentyl terephthalate | 2.00 |
| | dermofeel ® sensolv MB (Isoamyl Laurate) | 7.00 |
| | ABIL ® 350 (Dimethicone) | 0.50 |
| | TEGOSOFT ® CR (Cetyl Ricinoleate) | 2.00 |
| | Bis-ethylhexyloxyphenol Methoxyphenyl Triazine | 1.50 |
| | (Tinosorb S, BASF) | |
| | Butyl Methoxydibenzoylmethane | 1.00 |
| | Ethylhexyl Triazone | 0.75 |
| | Homomenthyl Salicylate | 1.50 |
| | Ethylhexyl Salicylate | 1.50 |
| | Octocrylene | 4.00 |
| | dermofeel ® TocoBalance (Tocopherol, Helianthus | 0.70 |
| | Annuus (Sunflower) Seed Oil) | |
| | TEGO ® Feel C 10 (Cellulose) | 2.00 |
| | TEGO ® SP 13-1 (Poly C10-30 Alkyl Acrylate) | 1.50 |
| | Phytosphingosine SLC (Salicyloyl Phytosphingosine) | 0.10 |
| B | Water | to 100 |
| | Glycerol | 3.00 |
| | EDTA | 0.05 |
| | TEGO ® Cosmo C 100 (Creatine) | 0.50 |
| C | Xanthan Gum (Ketrol CG-SFT, CP Kelco) | 0.50 |
| D | TEGO ® Pep Up (Tetrapeptide-4; Glycerin) | 2.00 |
| E | Verstatil ® PC (Phenoxyethanol, Caprylyl Glycol) | 1.20 |
| | Summer Holiday (Düllberg) | 0.10 |
| | Tromethamine (Trisaminomethane, 30% in water) | q.s. |

### PEG-Free AP/Deo Roll-On

| Phase | Raw material | w/w % |
|---|---|---|
| A | TEGO ® Care PS (Methyl Glucose Sesquistearate) | 1.75 |
| | Polyglyceryl-4 Laurate | 0.25 |
| | Di(iso)pentyl terephthalate | 4.00 |
| | TEGOSOFT ® E (PPG-15 Stearyl Ether) | 3.00 |
| | TEGO ® Cosmo P 813 (Polyglyceryl-3 Caprylate) | 1.00 |
| | dermofeel ® TEC eco (Triethyl Citrate) | 1.00 |
| | TEGODEO ® PY 88 G (Zinc Ricinoleate) | 1.00 |
| B | Propylene Glycol | 2.00 |
| | Water | to 100 |
| | Aluminium Chlorohydrate (50% in Water) | 15.00 |
| | Hydroxyethylcellulose (Natrosol 250HHR) | 0.75 |
| C | Dipropylene Glycol; Methylparaben; Ethylparaben; | 0.80 |
| | Aqua; Methylisothiazolinone (Microcare ® MEM, | |
| | Thor GmbH) | |
| | Perfume | q.s. |

### AP/Deo Stick

| Phase | Raw material | w/w % |
|---|---|---|
| A | TEGOSOFT ® APS (PPG-11 Stearyl Ether) | 5.00 |
| | Di(iso)pentyl terephthalate | 5.00 |
| | Stearyl Alcohol | 16.25 |
| | Hydrogenated Castor Oil | 1.75 |
| B | Cyclopentasiloxane (and) Cyclohexasiloxane | to 100 |
| C | AEROSIL R 972 V (Silica Dimethylsilylate) | 3.00 |
| | Aluminium Chlorohydrate (Micro Dry Ultrafine, Reheis) | 20.00 |
| D | TEGO ® Cosmo P 813 (Polyglyceryl-3 Caprylate) | 0.50 |

### W/O AP Aerosol Spray

| Phase | Raw material | w/w % |
|---|---|---|
| A | ABIL ® EM 90 (Cetyl PEG/PPG-10/1 Dimethicone) | 2.00 |
| | ISOLAN ® GPS (Polyglyceryl-4 Diisostearate/ | 2.00 |
| | Polyhydroxystearate/Sebacate) | |
| | Di(iso)pentyl terephthalate | 8.00 |
| | dermofeel ® sensolv (Isoamyl Laurate) | 25.00 |
| | Perfume | 3.00 |
| B | Water | to 100 |
| | Aluminium Chlorohydrate (Reach 501, Reheis) | 20.00 |
| | Propylene Glycol | 5.00 |
| C | Euxyl ® PE 9010 (Phenoxyethanol, Ethylhexylglycerin; | 0.80 |
| | Schülke & Mayr GmbH) | |

### AP/Deo Aerosol Spray (Water Free)

| Phase | Raw material | w/w % |
|---|---|---|
| A | Cyclopentasiloxane | to 100 |
| | Disteardimonium Hectorite | 3.00 |
| | Perfume | 5.00 |
| | Aerosil R 972 (Silica Dimethyl Silylate) | 2.00 |
| B | Aluminium Chlorohydrate (Activated Aloxicoll P) | 25.00 |
| | Di(iso)pentyl terephthalate | 8.00 |
| | dermosoft ® decalact (Sodium Caproyl/Lauroyl | 2.00 |
| | Lactylate) | |
| | Alcohol | 5.00 |

### O/W AP/Deo Roll-On

| Phase | Raw material | w/w % |
|---|---|---|
| A | TEGO ® Alkanol S 20 P (Steareth-20) | 1.00 |
| | TEGO ® Alkanol S 2 Pellets (Steareth-2) | 2.20 |
| | TEGOSOFT ® liquid (Cetearyl Ethylhexanoate) | 2.00 |
| | Di(iso)pentyl terephthalate | 2.00 |
| | ABIL ® 350 (Dimethicone) | 0.50 |
| | TEGODEO ® PY 88 G (Zinc Ricinoleate) | 1.00 |
| B | Glycerol | 3.00 |
| | Water | to 100 |
| C | Aluminium Chlorohydrate (50% in water; Reach 501 L, | 30.00 |
| | Reheis) | |
| | Preservative, Perfume | q.s. |

### PEG- and ACH-Free AP/Deo Roll-On

| Phase | Raw material | w/w % |
|---|---|---|
| A | TEGO ® Care APD 18 (Polyglyceryl-6 Stearate; | 5.00 |
| | Polyglyceryl-6 Behenate; C18-22 Hydroxyalkyl | |
| | Hydroxypropyl Guar) | |
| | Di(iso)pentyl terephthalate | 5.00 |
| B | Water | to 100 |
| | Glycerol | 3.00 |
| | Potassium Alum | 5.00 |
| C | Preservative, Perfume | q.s. |

1. A composition comprising:
A) at least one substance of the general formula (I) 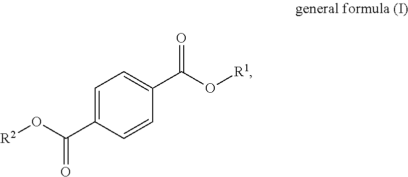
wherein R¹ and R² are each independently, the same or different, selected from the group consisting of organic radicals comprising 2 to 18 carbon atoms; and
B) at least one substance selected from the group consisting of UV light protection filter substances, and deodorant and antiperspirant active ingredients.

2. The composition according to claim 1, wherein component B) is a UV light protection filter substance comprising a triazine derivative.

3. The composition according to claim 1, wherein component B) is a UV light protection filter substance selected from the group consisting of butyl methoxydibenzoylmethane, ethylhexyl triazone, bis-ethylhexyloxyphenol methoxyphenyl triazine, diethylhexyl butamido triazone, diethylamino hydroxybenzoyl hexyl benzoate, benzophenone-3, and 4-methylbenzylidene camphor.

4. The composition according to claim 1, wherein component B) is a deodorant and antiperspirant active ingredient further comprising an astringent.

5. The composition, according to claim 1, wherein,
component A) is present in an amount of 0.1% by weight to 60% by weight, and
component B) is present in an amount of 0.1% by weight to 50% by weight.

6. A method of solubilizing a component in a cosmetic or pharmaceutical composition, comprising:
mixing at least one substance of the general formula (I), as a solubilizer or solvent, into a cosmetic or pharmaceutical composition; 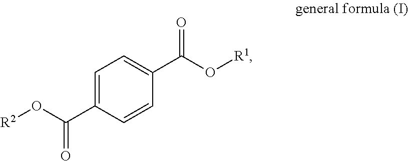
wherein R¹ and R² are each independently, the same or different, selected from the group consisting of organic radicals comprising 2 to 18 carbon atoms.

7. The composition according to claim 1, wherein in the general formula (I), R¹ and/or R² is an aliphatic radical comprising 2 to 18 carbon atoms.

8. The composition according to claim 1, wherein in the general formula (I), R¹ and/or R² is an alkyl radical comprising 2 to 18 carbon atoms.

9. The composition according to claim 1, wherein in the general formula (I), R¹ and/or R² is an alkyl radical comprising 4 to 8 carbon atoms.

10. The composition according to claim 4, wherein the astringent is at least one selected from the group consisting of an aluminium salt, a zinc salt, and an aluminium zirconium complex.

11. The composition according to claim 5, wherein the composition comprises component A) in an amount of 0.5% by weight to 30% by weight; and wherein the composition comprises component B) in an amount of 1% by weight to 40% by weight.

12. The composition according to claim 5, wherein the composition comprises component A) in an amount of 1% by weight to 20% by weight; and wherein the composition comprises component B) in an amount of 3% by weight to 30% by weight.

13. The method according to claim 6, wherein in the general formula (I), R¹ and/or R² is an aliphatic radical comprising 2 to 18 carbon atoms.

14. The method according to claim 6, wherein in the general formula (I), R¹ and/or R² is an alkyl radical comprising 2 to 18 carbon atoms.

15. The method according to claim 6, wherein in the general formula (I), R¹ and/or R² is an alkyl radical comprising 4 to 8 carbon atoms.

16. The method according to claim 6, wherein the cosmetic or pharmaceutical composition is a substance selected from the group consisting of UV light protection filter substances, and deodorant and antiperspirant active ingredients.
